(19) United States
(12) Patent Application Publication
(10) Pub. No.: US 2026/0240734 A1
(43) Pub. Date: Aug. 20, 2026

(54) Film Forming Oral Care Compositions And Methods For The Same

(71) Applicant: Colgate-Palmolive Company, New York, NJ (US)

(72) Inventors: Vivian NGUYEN, Somerset, NJ (US); Melissa MARTINETTI, Bridgewater, NJ (US)

(73) Assignee: Colgate-Palmolive Company, New York, NJ (US)

(21) Appl. No.: 19/470,880

(22) PCT Filed: Mar. 28, 2024
(86) PCT No.: PCT/US2024/021983

§ 371 Date: Sep. 30, 2025

Related U.S. Application Data
(60) Provisional application No. 63/494,561, filed on Apr. 6, 2023.

(51) Int. Cl. A61K 8/22; A61K 8/19; A61K 8/81; A61Q 11/00

(52) U.S. Cl. CPC A61K 8/22; A61K 8/19; A61K 8/8158; A61Q 11/00; A61K 2800/92; A61K 2800/95

(57) ABSTRACT

Film forming oral care compositions, films prepared therefrom and methods for the same are disclosed. The film forming oral care composition includes a whitening agent, an alkali metal carbonate or bicarbonate, a copolymer of acrylates and octylacrylamide and an orally acceptable solvent. The whitening agent includes an oxidizing agent and the copolymer is present in an amount less than 18 wt %, based on the total weight of the oral care composition. The method includes applying the film forming oral care composition to surfaces of teeth and evaporating at least a portion of the orally acceptable solvent to prepare a film on the surfaces of the teeth.

### CROSS-REFERENCE TO RELATED APPLICATION

This application claims priority to U.S. Provisional Application No. 63/494,561, entitled “Film Forming Oral Care Compositions and Methods for the Same,” filed on Apr. 6, 2023, the content of which is incorporated by reference in its entirety for all purposes.

### BACKGROUND

Many substances, such as food, tobacco products, and beverages (e.g., coffee) may often stain surfaces of teeth, thereby reducing the whiteness thereof. As such, conventional tooth whitening compositions are often utilized to deliver one or more whitening agents to the teeth. For example, conventional tooth whitening compositions, such as film forming tooth whitening compositions, may often include a combination of an active whitening agent and a polymer delivery system configured to deliver the active whitening agent to surfaces of the teeth to thereby whiten or reduce staining of the teeth. Certain whitening agents, such as peroxides, however, present formulation challenges due to their volatility and reactivity. Conventional tooth whitening compositions have addressed the volatility and reactivity of the whitening agents by utilizing relatively high concentrations of the polymer delivery system. For example, it was previously discovered that providing the polymer delivery system in concentrations of about 15 wt %, about 18 wt %, or more reduced the degradation of the whitening agents (e.g., peroxides). The relatively high concentrations of the polymer delivery system, however, resulted in the formation of films that were difficult to remove via brushing.

What is needed, then, are improved oral care products and film forming oral care compositions having reduced concentrations of the polymer delivery system, and methods for the same.

### BRIEF SUMMARY

This summary is intended merely to introduce a simplified summary of some aspects of one or more implementations of the present disclosure. Further areas of applicability of the present disclosure will become apparent from the detailed description provided hereinafter. This summary is not an extensive overview, nor is it intended to identify key or critical elements of the present teachings, nor to delincate the scope of the disclosure. Rather, its purpose is merely to present one or more concepts in simplified form as a prelude to the detailed description below.

The foregoing and/or other aspects and utilities embodied in the present disclosure may be achieved by providing a film forming oral care composition for whitening teeth. The film forming oral care composition may include a whitening agent, an alkali metal carbonate or bicarbonate, a copolymer of acrylates and octylacrylamide, and an orally acceptable solvent. The whitening agent may include an oxidizing agent present in an amount of from greater than 0 wt % to about 10 wt % or less, based on the total weight of the oral care composition. The copolymer may be present in an amount less than 18 wt %, based on the total weight of the oral care composition.

In at least one implementation, the oxidizing agent may include a peroxide compound, preferably, the peroxide compound comprises hydrogen peroxide.

In at least one implementation, the copolymer of acrylates and octylacrylamide may include 2-propenoic acid, 2-methyl-, 2-methylpropyl ester, 2-propenoic acid, N-(1,1,3,3-tetramethylbutyl)-2-propenamide (CAS 129702-02-9), preferably, the copolymer of acrylates and octylacrylamide includes DERMACRYL®.

In at least one implementation, the copolymer of acrylates and octylacrylamide may be present in an amount of from about 1 wt % to less than 18 wt %, preferably, about 1 wt % to about 10 wt %, more preferably, about 1 wt % to about 5 wt %, based on the total weight of the oral care composition.

In at least one implementation, the alkali metal carbonate or bicarbonate may include one or more of sodium carbonate, potassium carbonate, sodium bicarbonate, potassium bicarbonate, or a combination thereof, preferably, the alkali metal carbonate or bicarbonate includes sodium bicarbonate.

In at least one implementation, the alkali metal carbonate or bicarbonate may be present in an amount sufficient to interact synergistically with the whitening agent to prevent the degradation of the whitening agent. Preferably, the alkali metal carbonate or bicarbonate is present in an amount from about 0.1 wt % to about 1 wt %, about 0.15 wt % to about 0.8 wt %, about 0.2 wt % to about 0.5 wt %, based on the total weight of the oral care product or the film forming oral care composition thereof.

In at least one implementation, the alkali metal carbonate or bicarbonate and the whitening agent may be present in a synergistic weight ratio of from about 1:3 to about 1:75, preferably, a synergistic weight ratio of about 1:3, about 1:15, about 1:19, about 1:30, about 1:50, about 1:65, or about 1:75.

In at least one implementation, the orally acceptable vehicle may include one or more of ethanol, methanol, isopropanol, butanol, water, acetone, methyl ethyl ketone, ethyl acetate, butyl acetate, cyclohexanone, cyclohexane, methylene chloride, chloroform, carbon tetrachloride, trichloroethylene, perchloroethylene, methyl glycol acetate, toluene, benzene, diethyl ether, benzyl alcohol, or the like, or a combination thereof. Preferably, the orally acceptable vehicle includes ethanol.

In at least one implementation, the film forming oral care composition is free of cellulose and derivatives of cellulose.

In at least one implementation, the film forming oral care composition further includes an additional component comprising one or more of an anti-caries agent, an anti-calculus agent, an anti-inflammatory agent, a desensitizing agent, a viscosity modifier, a thickening agent, a diluent, a surface active agent, an orally acceptable surfactant, a pH modifying agent, a therapeutic agent, a humectant, a mouth feel agent, an enzyme, a sweetening agent, a flavor agent, preservatives, vitamins, pigments, or a combination thereof.

In at least one implementation, the whitening agent further includes a whitening pigment.

In at least one implementation, the film forming oral care composition may include a viscosity measured at about 25° C. of from about 1 cP to less than 60 cP, preferably, about 1 cP to about 15 cP, more preferably about 2 cP to about 11 cP.

In at least one implementation, the film forming oral care composition may consist of the whitening agent, the alkali metal carbonate or bicarbonate, the copolymer of acylates and octylacrylamide, the orally acceptable solvent, and optionally, the additional component.

The foregoing and/or other aspects and utilities embodied in the present disclosure may be achieved by providing a film prepared from any one or more of the film forming oral care compositions disclosed herein.

The foregoing and/or other aspects and utilities embodied in the present disclosure may be achieved by providing a method for whitening teeth. The method may include applying any one or more of the film forming oral care compositions disclosed herein to surfaces of the teeth. The method may also include evaporating at least a portion of the orally acceptable solvent to form a film on the surfaces of the teeth.

Further areas of applicability of the present disclosure will become apparent from the detailed description provided hereinafter. It should be understood that the detailed description and specific examples, while indicating some typical aspects of the disclosure, are intended for purposes of illustration only and are not intended to limit the scope of the disclosure.

### DETAILED DESCRIPTION

The following description of various typical aspect(s) is merely exemplary in nature and is in no way intended to limit the disclosure, its application, or uses.

As used throughout this disclosure, ranges are used as shorthand for describing each and every value that is within the range. It should be appreciated and understood that the description in a range format is merely for convenience and brevity, and should not be construed as an inflexible limitation on the scope of any embodiments or implementations disclosed herein. Accordingly, the disclosed range should be construed to have specifically disclosed all the possible subranges as well as individual numerical values within that range. As such, any value within the range may be selected as the terminus of the range. For example, description of a range such as from 1 to 5 should be considered to have specifically disclosed subranges such as from 1.5 to 3, from 1 to 4.5, from 2 to 5, from 3.1 to 5, etc., as well as individual numbers within that range, for example, 1, 2, 3, 3.2, 4, 5, etc. This applies regardless of the breadth of the range.

Unless otherwise specified, all percentages and amounts expressed herein and elsewhere in the specification should be understood to refer to percentages by weight. The amounts given are based on the active weight of the material.

Additionally, all numerical values are “about” or “approximately” the indicated value, and take into account experimental error and variations that would be expected by a person having ordinary skill in the art. It should be appreciated that all numerical values and ranges disclosed herein are approximate values and ranges, whether “about” is used in conjunction therewith. It should also be appreciated that the term “about,” as used herein, in conjunction with a numeral refers to a value that may be ±0.01% (inclusive), ±0.1% (inclusive), ±0.5% (inclusive), ±1% (inclusive) of that numeral, ±2% (inclusive) of that numeral, ±3% (inclusive) of that numeral, +5% (inclusive) of that numeral, ±10% (inclusive) of that numeral, or ±15% (inclusive) of that numeral. It should further be appreciated that when a numerical range is disclosed herein, any numerical value falling within the range is also specifically disclosed.

As used herein, “substantially free” of a material may refer to a composition, component, or phase where the material is present in an amount of less than 10.0 weight %, less than 5.0 weight %, less than 3.0 weight %, less than 1.0 weight %, less than 0.1 weight %, less than 0.05 weight %, less than 0.01 weight %, less than 0.005 weight %, or less than 0.0001 weight % based on a total weight of the composition, component, or phase. As used herein, “free” of a material refers to a composition, component, or phase where the material is excluded from the composition, component, or phase, respectively.

All references cited herein are hereby incorporated by reference in their entireties. In the event of a conflict in a definition in the present disclosure and that of a cited reference, the present disclosure controls.

The present inventors have surprisingly and unexpectedly discovered that oral care products or film forming oral care compositions thereof including a whitening agent and a combination of an alkali metal carbonate or bicarbonate, such as sodium bicarbonate, and a film forming polymer in amounts of less than 18 wt % or less than 15 wt %, were capable of maintaining the stability of and/or preventing or reducing the degradation of the whitening agent. The oral care product and the film forming composition prevented the degradation of the whitening agent for up to three months under accelerated aging conditions.

The present inventors have also surprisingly and unexpectedly discovered that oral care products or film forming oral care compositions thereof including a whitening agent and a combination of an alkali metal carbonate or bicarbonate, such as sodium bicarbonate, and a film forming polymer in amounts of less than 18 wt % or less than 15 wt %, maintained a clear, colorless liquid after exposure to accelerated aging conditions, thereby demonstrating the stability thereof. The oral care product and the film forming composition maintained the stability for up to three months under accelerated aging conditions.

The present inventors have further surprisingly and unexpectedly discovered that oral care products or film forming oral care compositions thereof including a whitening agent and a combination of an alkali metal carbonate or bicarbonate, such as sodium bicarbonate, and a film forming polymer in amounts of less than 18 wt % or less than 15 wt %, exhibited whitening efficacy comparable to oral care products or film forming oral care compositions including a whitening agent and a film forming polymer in amounts of about 18 wt % or greater, and excluding the alkali metal carbonate or bicarbonate.

### Compositions

Compositions disclosed herein may be or include an oral care product or a film forming oral care composition thereof. For example, the compositions disclosed herein may be an oral care product including the film forming oral care composition, or the film forming oral care composition thereof. In at least one implementation, the film forming oral care composition may include one or more of a whitening agent, at least one alkali metal carbonate or bicarbonate, one or more polymers having a hydrophilic component or functional group and a hydrophobic component or functional group, an orally acceptable vehicle, or a combination thereof. For example, the film forming oral care composition may include a peroxide as the whitening agent, sodium bicarbonate as the at least one alkali metal carbonate, a polymer having an acrylate or acrylic acid component (i.e., hydrophilic component) and a bulky hydrophobic component, such as a tert-octylacrylamide, a butylaminoethyl methacrylate, and/or a styrene, and ethanol as the orally acceptable vehicle.

As further described herein, the film forming oral care compositions disclosed herein or the films formed therefrom may be capable of or configured to provide improved whitening efficacy to surfaces of the oral cavity, such as surfaces of teeth, as compared to film forming oral care compositions including the polymer in an amount greater than or equal to 15 wt % or greater than or equal to 18 wt %. The film forming oral care compositions disclosed herein or the films formed therefrom may also be capable of or configured to provide improved delivery of a whitening agent to surfaces of the teeth. The film forming oral care composition disclosed herein may also be capable of or configured to decrease, reduce, or otherwise inhibit the degradation of the whitening agent. The film forming oral care compositions disclosed herein may also be capable of or configured to decrease, reduce, or otherwise inhibit the degradation of the whitening agent while maintaining the one or more polymers in an amount less than or equal to about 15 wt %, less than or equal to about 10 wt %, less than or equal to about 5 wt %, less than or equal to about 3 wt %, less than or equal to about 1 wt %, or less.

The oral care product or the film forming oral care composition thereof may be prepared in the form of a flowable or liquid composition, such as a viscous liquid composition. The oral care product or the film forming oral care composition may be utilized to prepare or form a film. For example, the orally acceptable vehicle (e.g., ethanol) of the oral care product or the film forming oral care composition thereof may be at least partially evaporated to form the film. The oral care product or the film forming oral care composition thereof may be utilized to form a film on surfaces of the oral cavity, such as surfaces of teeth, to whiten the surfaces of the teeth. The film may not be visible to the naked or unaided eye in ambient light when formed on surfaces of the teeth. For example, the films prepared from oral care products or film forming oral care compositions disclosed herein having about 10 wt % or less of the polymer, about 5 wt % or less of the polymer, or about 1 wt % or less of the polymer are not visible, as compared to films prepared from oral care products or film forming oral care compositions having about 18 wt % of the polymer or more.

The oral care product or the film forming oral care composition thereof may have a viscosity measured at about 25° C. of from about 1 cP to less than 60 cP. For example, the oral care product or the film forming oral care composition thereof may have a viscosity measured at about 25° C. of from about 1 cP, about 2 cP, about 3 cP, about 4 cP, about 5 cP, about 10 cP, about 15 cP, or about 20 cP to about 25 cP, about 30 cP, about 35 cP, or about 50 cP. In a preferred implementation, the oral care product or the film forming oral care composition thereof may have a viscosity measured at about 25° C. of from about 1 cP to about 15 cP, more preferably about 2 cP to about 11 cP.

### Polymers

The one or more polymers of the film forming oral care composition may be or include, but are not limited to, one or more film forming polymers. As used herein, the expression “film forming polymer” may refer to or encompass polymers, prepolymers, and/or monomers capable of or configured to forming, either alone or in the presence of at least one additional agent, a continuous film on a substrate, such as the surfaces of the oral cavity (e.g., surface of teeth).

The film forming polymer may include one or more hydrophilic components or functional groups and/or one or more hydrophobic components or functional groups. The hydrophilic components or functional groups may be capable of or configured to facilitate, maintain, or increase the adhesion of the film forming polymer to surfaces of the oral cavity. Illustrative hydrophilic components or functional groups may be or include, but are not limited to, acrylate or acrylic acid groups, carboxyl groups, carbonyl groups, sulfhydryl groups, phosphate groups, hydroxyl groups, amines, disulfides, nitro groups, or the like, or a combination thereof. The hydrophilic components may be provided by one or more acrylate monomers. Illustrative acrylate monomers may include, but are not limited to, methacrylates, methyl acrylate, ethyl acrylate, 2-chloroethyl vinyl ether, 2-ethylhexyl acrylate, hydroxyethyl methacrylate, butyl acrylate, butyl methacrylate, trimethylolpropane triacrylate, or the like, or a combination thereof.

The hydrophobic components or functional groups of the film forming polymer may be capable of or configured to provide one or more benefits or properties to the film formed by the film forming polymer. For example, the hydrophobic components may be capable of or configured to provide, maintain, or increase the efficacy of the film for delivering the whitening agent to surfaces of the teeth. In another example, the hydrophobic component may be capable of or configured to form a film capable of maintaining sufficient contact with the surfaces of the teeth for a sufficient amount of time. Illustrative hydrophobic components or functional groups may be or include, but are not limited to, an alkyl chain, a polyethylene glycol, a polypropylene, a polyester, a polyorthoester, a phospholipid, a long chain fatty acid, a vinyl chloride, fluorethylene, a siloxane, a urethane, an octylacrylamide, a butylaminoethyl, a styrene, or the like, or a mixture or combination thereof.

In at least one implementation, the film forming polymer may be or include a homopolymer. In another implementation, the film forming polymer may be or include a copolymer composed of or produced by two or more dissimilar monomers. For example, the film forming polymer may be or include a copolymer composed of a first monomer capable of or configured to provide the hydrophilic component or functional group and a second monomer capable of or configured to provide the hydrophobic component or functional group.

In at least one implementation, the film forming polymer may be or include hydrophobic copolymer. For example, the film forming polymer may be or include a carboxylated acrylic copolymer. For example, the film forming polymer may be a copolymer of octylacrylamide and one or more monomers, where the one or more monomers may include one or more of acrylic acid, methacrylic acid, and any one or more simple esters thereof. In another example, the film forming polymer may be a polymer formed from octylacrylamide, t-butylaminoethyl methacrylate, and one or more monomers of acrylic acid, methacrylic acid, or any one or more simple esters thereof.

Illustrative film forming polymers may be or include, but are not limited to, those sold under the trade names DERMACRYL®, AMPHOMER®, BALANCER, and VERSACRYL®, which are commercially available from AkzoNobel Company, Surface Chemistry of Amsterdam, Netherlands. For example, the film forming polymer may be or include, but are not limited to, AMPHOMER® 4961, AMPHOMER® HC, DERMACRYL® 2.0, RESYN™ XP, a film forming polymer selected from octylacrylamide/acrylates/butylaminoethyl methacrylate copolymer, such as AMPHOMER® LV-71, AMPHOMER®, AMPHOMER® EDGE™, BALANCE® 47, or the like, or a combination thereof, all of which are commercially available from AkzoNobel Company, Surface Chemistry of Amsterdam, Netherlands. The film forming polymer may be selected from vinyl acetate/butyl maleate/isobornyl acrylate copolymers, such as ADVANTAGE™ PLUS from Ashland Global Specialty Chemicals, Inc. of Covington, KY. The film forming polymer may also be selected from acrylates/t-butylacrylamide copolymers, such as ULTRAHOLD® STRONG and ULTRAHOLD®8 from BASF SE of Ludwigshafen, Germany. The film forming polymer may also be selected from acrylates/dimethylaminoethyl methacrylate copolymers, such as the EUDRAGIT® range of polymers from Evonik Industries of Essen, Germany, such as EUDRAGIT®E100, EUDRAGIT® E PO (CAS: 24938-16-7), EUDRAGIT® RS 100, EUDRAGIT® RS PO, EUDRAGIT® RL PO, EUDRAGIT® RL 100, or the like, or a combination thereof. For example, the film forming polymer may be poly(butyl methacrylate-co-(2-dimethylaminoethyl) methacrylate-co-methyl methacrylate) 1:2:1. The film forming polymer may be selected from polyvinylpyrrolidone/vinyl acetate, such as the PVP/VA series of polymers, and triacontanyl PVP, such as GANEX™ WP-660, both of which are commercially available from Ashland Global Specialty Chemicals Inc. of Covington, KY. The film forming polymer may be selected from at least one of octylacrylamide/acrylates/butylaminoethyl methacrylate copolymer, vinyl acetate (VA)/butyl maleate/isobornyl acrylate copolymer, acrylates/t-butylacrylamide copolymer, polyvinylpyrrolidone/vinyl acetate copolymer, triacontanyl PVP copolymer, acrylates/dimethylaminoethyl methacrylate copolymer, or the like, or a combination thereof. In a preferred implementation, the film forming polymer is a copolymer of acrylates and octylacrylamide. For example, the film forming polymer may be or include copolymer 2-propenoic acid, 2-methyl-, 2-methylpropylester, polymer with 2-propenoic acid and N-(1,1,3,3-tetramethylbutyl)-2-propenamide or 2-propenoic acid, 2-methyl-, 2-methylpropyl ester, 2-propenoic acid, N-(1,1,3,3-tetramethylbutyl)-2-propenamide (CAS 129702-02-9). For example, the film forming polymer may be or include, but is not limited to, DERMACRYL® 79, which is commercially available from AkzoNobel Company, Surface Chemistry of Amsterdam, Netherlands. In another preferred implementation, the film forming polymer may be an octylacrylamide/acrylates/butylaminoethyl methacrylate copolymer. For example, the film forming polymer may be or include copolymer 2-Propenoic acid, 2-methyl-, 2-(1,1-dimethylethyl)aminoethyl ester, polymer with methyl 2-methyl-2-propenoate, 1,2-propanediol mono(2-methyl-2-propenoate), 2-propenoic acid and N-(1,1,3,3-tetramethylbutyl)-2-propenamide (CAS 70801-07-9). For example, the film forming polymer may be or include, but is not limited to AMPHOMER® LV-71, commercially available from AkzoNobel Company, Surface Chemistry. In yet another preferred implementation, the film forming polymer may be EUDRAGIT® E PO (CAS: 24938-16-7).

In at least one implementation, the film forming polymer may include cellulose and/or a derivative of cellulose or cellulose derivative, such as an alkyl cellulose ether. In a preferred implementation, the film forming polymer may be free or substantially free of cellulose and/or a derivative of cellulose or cellulose derivative, such as an alkyl cellulose ether. As used herein, the expression “alkyl cellulose ether” may refer to a lower alkyl ether of cellulose, such as an ethyl cellulose.

The amount or concentration of any one or more of the film forming polymers present in the oral care product or the film forming oral care composition thereof may vary widely. In at least one implementation, the amount of any one or more of the film forming polymers present may be an amount effective to form a film capable of or configured to deliver the whitening agent to surfaces of the teeth. The amount of any one or more of the film forming polymers may also be present in an amount effective to form a film capable of or configured to maintain contact with the surfaces of the teeth for a sufficient amount of time to whiten teeth while being readily removed (e.g., via abrading, brushing, and/or rinsing). The amount of any one or more of the film forming polymer may be present in an amount greater than 0 wt % and less than or equal to 20 wt %, less than or equal to 18 wt %, less than or equal to 16 wt %, less than or equal to 15 wt %, less than or equal to 14 wt %, less than or equal to 13 wt %, less than or equal to 10 wt %, less than or equal to 5 wt %, or less than or equal to 1 wt %, based on the total weight of the oral care product or the film forming oral care composition thereof. The amount of any one or more of the film forming polymer may be present in an amount of from about 1 wt % to about 18 wt %, preferably, about 1 wt % to about 10 wt %, more preferably, about 1 wt % to about 5 wt %, based on the total weight of the oral care product or the film forming oral care composition thereof.

### Whitening Agent

As discussed above, the film forming oral care composition may include one or more whitening agents. The whitening agent may be an active ingredient. As used herein, the expression “whitening agent” may refer to a material that affects the whitening of surfaces of the teeth to which it is applied. Any whitening agent known or developed in the art may be used in the present film forming oral care composition. Illustrative whitening agents may be or include, but are not limited to, a whitening pigment, an oxidizing agent, a reducing agent, a peroxide compound, a whitening complex, or a combination thereof. As used herein, the expression “whitening complex” may refer to a whitening agent as described herein complexed with a polymer or copolymer that is capable of or configured to release the whitening agent upon exposure to highly aqueous environments, such as in the oral cavity. In a preferred implementation, the whitening agent includes a peroxide compound, more preferably, hydrogen peroxide.

The whitening pigment may include particles ranging in sizes from about 0.1 μm to about 10 μm with a refractive index greater than about 1.2. Illustrative whitening pigments may be or include, but are not limited to, titanium dioxide particles, zinc oxide particles, aluminum oxide particles, tin oxide particles, calcium oxide particles, magnesium oxide particles, barium oxide particles, silica particles, zirconium silicate particles, mica particles, talc particles, tetracalcium phosphate particles, amorphous calcium phosphate particles, alpha-tricalcium phosphate particles, beta-tricalcium phosphate particles, hydroxyapatite particles, calcium carbonate particles, zinc phosphate particles, silicon dioxide particles, zirconium silicate particles, or a combination thereof. The whitening pigment, such as titanium dioxide particles, may be present in an amount that is sufficient or effective to whiten the teeth.

As noted above, the whitening agent may be an oxidizing agent, a reducing agent, or a combination thereof. As used herein, the expression “oxidizing agent” may refer to material and/or compounds that can accept an electron from another material and/or compound (e.g., molecule) in the environment of the oral cavity without having a deleterious or unacceptably harmful effect on the oral cavity in normal and accepted use. Illustrative oxidizing agents suitable for use with the oral care product or the film forming oral care composition may include, but are not limited to, peroxides, chlorites and hypochlorites. Examples of suitable chlorites and hypochlorites include those having alkali or alkaline metal cations and include calcium chlorite, barium chlorite, magnesium chlorite, lithium chlorite, sodium chlorite, potassium chlorite, calcium hypochlorite, barium hypochlorite, magnesium hypochlorite, lithium hypochlorite, lithium hypochlorite, and sodium hypochlorite.

In a preferred implementation, the whitening agent includes a peroxide compound. As used herein, the expression “peroxide compound” may refer to any compound including a bivalent oxygen-oxygen group. Peroxide compounds include peroxides and hydroperoxides, such as hydrogen peroxide, peroxides of alkali and alkaline earth metals, organic peroxy compounds, peroxy acids, pharmaceutically acceptable salts thereof, or a mixture or combination thereof. Peroxides of alkali and alkaline earth metals include lithium peroxide, potassium peroxide, sodium peroxide, magnesium peroxide, calcium peroxide, barium peroxide, or a mixture or combination thereof. Organic peroxy compounds include carbamide peroxide (also known as urea hydrogen peroxide), glyceryl hydrogen peroxide, alkyl hydrogen peroxides, dialkyl peroxides, alkyl peroxy acids, peroxy esters, diacyl peroxides, benzoyl peroxide, monoperoxyphthalate, or a mixture or combination thereof. Peroxy acids and their salts include organic peroxy acids, such as alkyl peroxy acids, monoperoxyphthalate, or a mixture thereof, as well as inorganic peroxy acid salts, such as persulfate, dipersulfate, percarbonate, perphosphate, perborate, and persilicate salts of alkali and alkaline earth metals, such as lithium, potassium, sodium, magnesium, calcium, and barium, or a mixture thereof. In a preferred implementation, the whitening agent includes a peroxide compound, preferably, hydrogen peroxide, more preferably, a solution of hydrogen peroxide. For example, the whitening agent may preferably be a 35% aqueous H₂O₂ solution or a 50% aqueous H₂O₂ solution.

As used herein, a “complex” is an entity formed by a loose association involving two or more molecular entities (ionic or uncharged), e.g., a whitening agent and a polymer. Illustrative whitening complexes may be or include, but are not limited to, polyvinylpyrrolidone-H₂O₂ complexes (PVP-H₂O₂). Polyvinylpyrrolidone is also known as poly-N-vinyl-poly-2-pyrrolidone and commonly abbreviated to “PVP”. PVP generally refers to a polymer containing vinylpyrrolidone (also referred to as N-vinylpyrrolidone, N-vinyl-2-pyrrolidione and N-vinyl-2-pyrrolidinone) as a monomeric unit. The monomeric unit includes a polar amide group, four non-polar methylene groups, and a non-polar methane group. Both linear and cross-linked complexes of PVP-H₂O₂ are known in the art, and PVP-H₂O₂ is considered to be stable in an anhydrous environment. Upon exposure to highly aqueous environments, such as in the oral cavity, the PVP-H₂O₂ dissociates into individual species (PVP polymer and H₂O₂). In one preferred implementation, the PVP-H₂O₂ complex, if present, is 80% by weight polyvinylpyrrolidone and 20% by weight H₂O₂.

The whitening agent may be present in the oral care product or the film forming oral care composition thereof in an amount sufficient to whiten teeth without damaging the oral cavity or surfaces thereof. In at least one implementation, the whitening agent may be present in the oral care product or the film forming oral care composition thereof in an amount of from greater than 0 wt %, greater than or equal to about 3 wt %, greater than or equal to about 6 wt %, greater than or equal to about 10 wt %, or greater than or equal to about 15 wt %, based on the total weight of the oral care composition. In another example, the whitening agent may be present in the oral care product or the film forming oral care composition thereof in an amount of from about 1 wt % to about 15 wt %, about 3 wt % to about 10 wt %, or about 6 wt %, based on the total weight of the oral care composition. As discussed above, the preferred whitening agent is hydrogen peroxide, which may be present in an amount of greater than 0 wt % to about 3 wt % or less, about 6 wt % or less, about 10 wt % or less, or about 15 wt % or less, based on the total weight of the oral care composition or the film forming oral care composition thereof.

### Alkali Metal Carbonate or Bicarbonate

The alkali metal carbonate or bicarbonate may be capable of or configured to interact with one or more components of the oral care product or the film forming oral care composition thereof to improve whitening of surfaces of the oral cavity, improve delivery of a whitening agent to surfaces of the teeth, decrease, reduce, or otherwise inhibit the degradation of the whitening agent (i.e., hydrogen peroxide), and/or at least partially stabilize the whitening agent. For example, as further demonstrated in the Examples herein, the combination of an alkali metal carbonate or bicarbonate and hydrogen peroxide in a film forming oral care composition including less than 18 wt % or less than 15 wt % of the polymers provides whitening efficacy comparable to a film forming oral care composition including at least 15 wt % or at least 18 wt % of the polymers and excluding the alkali metal carbonate or bicarbonate. It should be appreciated that conventional oral care products or film forming oral care compositions thereof include the polymers in relatively high concentrations, such as about 15 wt % or greater or about 18 wt % or greater, to reduce the degradation of the whitening agent. Accordingly, providing an oral care composition having relatively lower amounts of the polymer (e.g., less than or equal to about 15 wt %, less than or equal to about 18 wt %, or less) while concurrently providing the ability to preserve or prevent the degradation of the whitening agent is a non-obvious, surprising, and unexpected improvement over conventional oral care compositions that are difficult to remove (e.g., via brushing) due to the high concentrations of the polymer. Illustrative alkali metal carbonate or bicarbonate may be or include, but are not limited to, sodium carbonate, potassium carbonate, sodium bicarbonate, potassium bicarbonate, or the like, or a mixture or combination thereof. In a preferred implementation, the alkali metal carbonate includes at least sodium bicarbonate.

The one or more alkali metal carbonates or bicarbonates may be present in the oral care product or the film forming oral care composition thereof in an amount sufficient to interact with the one or more whitening agents and/or the polymer. For example, the one or more alkali metal carbonates or bicarbonates may be present in the oral care product or the film forming oral care composition thereof in an amount of from greater than 0 wt % to about 1 wt %, based on the total weight of the oral care product or the film forming oral care composition thereof. For example, the one or more alkali metal carbonates or bicarbonates may be present in the oral care product or the film forming oral care composition thereof in an amount of from about 0.1 wt %, about 0.15 wt %, about 0.2 wt %, about 0.3 wt %, or about 0.4 wt % to about 0.5 wt %, about 0.6 wt %, about 0.7 wt %, about 0.8 wt %, or about 1 wt %, based on the total weight of the oral care product or the film forming oral care composition thereof. In another example, the one or more alkali metal carbonates or bicarbonates may be present in the oral care product or the film forming oral care composition thereof in an amount of about 0.1 wt %, about 0.155 wt %, about 0.2 wt %, or about 1.0 wt %, based on the total weight of the oral care product or the film forming oral care composition thereof. In a preferred implementation, the one or more alkali metal carbonates or bicarbonates includes sodium bicarbonate in an amount of about 0.1 wt %, about 0.155 wt %, about 0.2 wt %, or about 1.0 wt %, based on the total weight of the oral care product or the film forming oral care composition thereof.

In at least one implementation, the one or more alkali metal carbonates or bicarbonates and the whitening agent may be present in a synergistic weight ratio of from about 1:3 (i.e., about 1 to about 3) to about 1:75. For example, the one or more alkali metal carbonates or bicarbonates and the whitening agent may be present in a synergistic weight ratio of about 1:3, about 1:15, about 1:19, about 1:30, about 1:50, about 1:65, or about 1:75.

### Orally Acceptable Vehicle

In at least one implementation, the oral care product or the film forming oral care composition thereof may include or be dispersed or dissolved in an orally acceptable vehicle. As used herein, the expression “orally acceptable vehicle” may refer to a suitable vehicle, ingredient, or combination of ingredients, which can be used to form and/or apply the film forming oral care composition or one or more components thereof to surfaces of the oral cavity in a safe and effective manner. For example, the orally acceptable vehicle may be a suitable solvent, such as a polar solvent, and the film forming oral care composition may be dispersed, dissolved, mixed, or otherwise contacted with the suitable solvent to prepare or form the oral care product. In another example, the orally acceptable vehicle may be a suitable solvent, such as a polar solvent, and the components of the film forming oral care composition may be dispersed, dissolved, mixed, or otherwise contacted with the suitable solvent to prepare or form the film forming oral care composition. In a preferred implementation, the solvent is a polar solvent capable of dissolving or dispersing the polymers of the oral care product or the film forming oral care composition thereof.

Illustrative orally acceptable vehicles or solvents thereof may be or include, but are not limited to, ethanol, methanol, isopropanol, butanol, water, acetone, methyl ethyl ketone, ethyl acetate, butyl acetate, cyclohexanone, cyclohexane, methylene chloride, chloroform, carbon tetrachloride, trichloroethylene, perchloroethylene, methyl glycol acetate, toluene, benzene, diethyl ether, benzyl alcohol, or the like, or a combination thereof. Preferably, the orally acceptable vehicle or the solvent thereof is selected such that it does not react with, or minimally reacts with, the whitening agent. For example, some orally acceptable vehicles or the solvents thereof are known to decompose hydrogen peroxide; and thus, would reduce the shelf-life of the whitening oral care product or the oral care composition disclosed herein. However, decomposition of the hydrogen peroxide does not necessarily result from reaction with some polar solvents. For example, peroxides may undergo self-decomposition. Nonetheless, incompatible solvents in the oral care composition may accelerate the self-decomposition of hydrogen peroxide. Accordingly, acceptable ingredients, including the polar solvent(s), are those that form an oral care product or a whitening film forming oral care composition thereof capable of retaining at least 60%, at least 80%, at least 90%, at least 95%, or greater of the peroxide active upon exposure to accelerated aging conditions. In a preferred implementation, the orally acceptable vehicle is or includes ethanol.

The orally acceptable vehicle may make up the balance of the oral care product or the film forming oral care composition thereof. In at least one implementation, the orally acceptable vehicle (e.g., ethanol) may be present in an amount of at least 60 weight %, at least 62 weight %, at least 64 weight %, at least 66 weight %, at least 68 weight %, at least 70 weight %, at least 72 weight %, at least 74 weight %, at least 76 weight %, at least 78 weight %, at least 80 weight %, at least 82 weight %, at least 84 weight %, at least 86 weight %, at least 88 weight %, at least 90 weight %, at least 92 weight %, at least 94 weight %, at least 96 weight %, at least 98 weight %, or at least 99 weight %, based on a total weight of the oral care product.

### Additional Ingredients

It should be appreciated by one having ordinary skill in the art, that the oral care products and/or the film forming oral care compositions thereof may include other additional ingredients/components. For example, the oral care products and/or the film forming oral care compositions thereof may include one or more anti-caries agents, anti-calculus agents, anti-inflammatory agents, desensitizing agents, viscosity modifiers, thickening agents, diluents, surface active agents (e.g., emulsifiers, foam modulators, etc.), orally acceptable surfactant, pH modifying agents (e.g., acids and bases), therapeutic agents (e.g., stannous ion, stannous ion sources, anti-microbial agents, calcium ion sources, zinc ion sources, potassium ion sources, etc.), a fluoride ion source or fluoride compound, humectants, mouth feel agents, enzymes, sweetening agents, flavor agents, colorants, preservatives, vitamins, pigments, or the like, or a combination thereof. It should further be appreciated by one having ordinary skill in the art that while general attributes of each of the above categories of materials may differ, there may be some common attributes and any given material may serve multiple purposes within two or more of such categories of materials.

In an exemplary implementation, the one or more additional ingredients includes a fluoride ion source or fluoride compound. Illustrative fluoride ion sources and fluoride compounds may be or include, but are not limited to, a soluble salt of a fluoride ion, sodium fluoride, potassium fluoride, calcium fluoride, zinc fluoride, zinc ammonium fluoride, lithium fluoride, ammonium fluoride, stannous fluoride, stannous fluorozirconate, or the like, or a combination thereof. In a preferred implementation, the fluoride compound includes sodium fluoride.

Any orally acceptable surfactant, most of which are anionic, nonionic or amphoteric, may be utilized. Suitable anionic surfactants include, but are not limited to, water-soluble salts of C₈₋₂₀ alkyl sulfates, sulfonated monoglycerides of C₈₋₂₀ fatty acids, sarcosinates, taurates and the like. Illustrative examples of these and other classes include, but are not limited to, sodium lauryl sulfate, sodium cocoyl monoglyceride sulfonate, sodium lauryl sarcosinate, sodium lauryl isoethionate, sodium laureth carboxylate, and sodium dodecyl benzenesulfonate. Suitable nonionic surfactants include, but are not limited to, poloxamers, polyoxyethylene sorbitan esters, fatty alcohol ethoxylates, alkylphenol ethoxylates, tertiary amine oxides, tertiary phosphine oxides, dialkyl sulfoxides, and the like. Suitable amphoteric surfactants include, but are not limited to, derivatives of C₈₋₂₀ aliphatic secondary and tertiary amines having an anionic group such as carboxylate, sulfate, sulfonate, phosphate, or phosphonate. A suitable example is cocoamidopropyl betaine.

All ingredients for use in the compositions described herein should be orally acceptable. As used herein, “orally acceptable” may refer to any ingredient that is present in a composition as described in an amount and form that does not render the composition unsafe for use in the oral cavity.

### Methods

The present disclosure may provide methods for preparing the oral care product or the film forming oral care composition thereof. The method may include mixing, stirring, combining, or otherwise contacting the whitening agent, the at least one alkali metal carbonate or bicarbonate, the one or more polymers, the orally acceptable vehicle, or a combination thereof with one another. For example, the method may include contacting hydrogen peroxide, sodium bicarbonate, an acrylates/octylacrylamide copolymer, ethanol, or a combination thereof with one another. In another example, the method may include contacting hydrogen peroxide, sodium bicarbonate, the copolymer 2-propenoic acid, 2-methyl-, 2-methylpropylester, polymer with 2-propenoic acid and N-(1,1,3,3-tetramethylbutyl)-2-propenamide (CAS 129702-02-9), ethanol, or a combination thereof with one another. Any one of the foregoing methods may further include contacting one or more of additional ingredients/components disclosed herein with components of the oral care product or the film forming oral care composition.

The present disclosure may also further provide methods for decreasing, reducing, or otherwise inhibiting the degradation of a whitening agent of an oral care product or a film forming oral care composition thereof. The method may include mixing, stirring, combining, or otherwise contacting the whitening agent, the at least one alkali metal carbonate or bicarbonate, the one or more polymers, the orally acceptable vehicle, or a combination thereof with one another. For example, the method may include contacting hydrogen peroxide, sodium bicarbonate, an acrylates/octylacrylamide copolymer, ethanol, or a combination thereof with one another. In another example, the method may include contacting hydrogen peroxide, sodium bicarbonate, the copolymer 2-propenoic acid, 2-methyl-, 2-methylpropylester, polymer with 2-propenoic acid and N-(1,1,3,3-tetramethylbutyl)-2-propenamide (CAS 129702-02-9), ethanol, or a combination thereof with one another. The sodium bicarbonate may be present in an amount sufficient to decrease, reduce, or otherwise inhibit the degradation of the hydrogen peroxide.

The present disclosure may further provide methods for whitening surfaces of an oral cavity, such as surfaces of teeth, of a human or other animal subject. The method may include contacting the oral care product or the film forming oral care composition thereof with the surfaces of the teeth. Contacting the oral care product or the film forming oral care composition thereof may include utilizing an applicator including the oral care product or the film forming oral care composition thereof to directly apply or contact the oral care product or the film forming oral care composition thereof to the surfaces of the teeth. The applicator may be or include, but is not limited to, a brush, a pen, a marker, or the like. The method may include forming a film on the surfaces of the teeth with the oral care product or the film forming oral care composition thereof. Forming the film on the surfaces of the teeth may include at least partially evaporating the orally acceptable vehicle (e.g., ethanol). The oral care product or the film forming oral care composition thereof may be contacted with the teeth for a plurality of minutes and/or hours. The method may include applying the oral care product or the film forming oral care composition thereof to the surfaces of the teeth once a day for a plurality of days.

The following numbered paragraphs disclose one or more exemplary variations of the subject matter of the application:

1. A film forming oral care composition for whitening teeth, the film forming oral care composition comprising: a whitening agent, wherein the whitening agent comprises an oxidizing agent present in an amount of from greater than 0 wt % to about 10 wt % or less, based on the total weight of the oral care composition; an alkali metal carbonate or bicarbonate; a copolymer of acrylates and octylacrylamide, wherein the copolymer is present in an amount less than 18 wt %, based on the total weight of the oral care composition; an orally acceptable solvent.

2. The film forming oral care composition of paragraph 1, wherein the oxidizing agent comprises a peroxide compound, preferably, the peroxide compound comprises hydrogen peroxide.

3. The film forming oral care composition of paragraph 1 or 2, wherein the copolymer of acrylates and octylacrylamide comprise 2-propenoic acid, 2-methyl-, 2-methylpropyl ester, 2-propenoic acid, N-(1,1,3,3-tetramethylbutyl)-2-propenamide (CAS 129702-02-9), preferably, the copolymer of acrylates and octylacrylamide comprises DERMACRYL®.

4. The film forming oral care composition of any one of the foregoing paragraphs, wherein the copolymer of acrylates and octylacrylamide is present in an amount of from about 1 wt % to less than 18 wt %, preferably, about 1 wt % to about 10 wt %, more preferably, about 1 wt % to about 5 wt %, based on the total weight of the oral care composition.

5. The film forming oral care composition of any one of the foregoing paragraphs, wherein the alkali metal carbonate or bicarbonate comprises one or more of sodium carbonate, potassium carbonate, sodium bicarbonate, potassium bicarbonate, or a combination thereof, preferably, the alkali metal carbonate or bicarbonate comprises sodium bicarbonate.

6. The film forming oral care composition of any one of the foregoing paragraphs, wherein the alkali metal carbonate or bicarbonate is present in an amount sufficient to interact synergistically with the whitening agent to prevent the degradation of the whitening agent, preferably, the alkali metal carbonate or bicarbonate is present in an amount from about 0.1 wt % to about 1 wt %, about 0.15 wt % to about 0.8 wt %, about 0.2 wt % to about 0.5 wt %, based on the total weight of the oral care product or the film forming oral care composition thereof.

7. The film forming oral care composition of any one of the foregoing paragraphs, wherein the alkali metal carbonate or bicarbonate and the whitening agent are present in a synergistic weight ratio of from about 1:3 to about 1:75, preferably, a synergistic weight ratio of about 1:3, about 1:15, about 1:19, about 1:30, about 1:50, about 1:65, or about 1:75.

8. The film forming oral care composition of any one of the foregoing paragraphs, wherein the orally acceptable vehicle comprises one or more of ethanol, methanol, isopropanol, butanol, water, acetone, methyl ethyl ketone, ethyl acetate, butyl acetate, cyclohexanone, cyclohexane, methylene chloride, chloroform, carbon tetrachloride, trichloroethylene, perchloroethylene, methyl glycol acetate, toluene, benzene, diethyl ether, benzyl alcohol, or the like, or a combination thereof, preferably, the orally acceptable vehicle comprises ethanol.

9. The film forming oral care composition of any one of the foregoing paragraphs, wherein the film forming oral care composition is free of cellulose and derivatives of cellulose.

10. The film forming oral care composition of any one of the foregoing paragraphs, further comprising an additional component comprising one or more of an anti-caries agent, an anti-calculus agent, an anti-inflammatory agent, a desensitizing agent, a viscosity modifier, a thickening agent, a diluent, a surface active agent, an orally acceptable surfactant, a pH modifying agent, a therapeutic agent, a humectant, a mouth feel agent, an enzyme, a sweetening agent, a flavor agent, preservatives, vitamins, pigments, or a combination thereof.

11. The film forming oral care composition of any one of the foregoing paragraphs, wherein the whitening agent further comprises a whitening pigment.

12. The film forming oral care composition of any one of the foregoing paragraphs, wherein the film forming oral care composition comprises a viscosity measured at about 25° C. of from about 1 cP to less than 60 cP, preferably, about 1 cP to about 15 cP, more preferably about 2 cP to about 11 cP.

13. The film forming oral care composition of any one of the foregoing paragraphs, wherein the film forming oral care composition consists of the whitening agent, the alkali metal carbonate or bicarbonate, the copolymer of acylates and octylacrylamide, the orally acceptable solvent, and optionally, the additional component.

14. A film prepared from the film forming oral care composition of any one of the foregoing paragraphs.

15. A method for whitening teeth, the method comprising: applying the film forming oral care composition of any one of paragraphs 1 to 13 to surfaces of the teeth; and evaporating at least a portion of the orally acceptable solvent to form a film on the surfaces of the teeth.

### EXAMPLES

The examples and other implementations described herein are exemplary and not intended to be limiting in describing the full scope of compositions and methods of this disclosure. Equivalent changes, modifications and variations of specific implementations, materials, compositions, and methods may be made within the scope of the present disclosure, with substantially similar results.

### Example 1

The stability of varying oral care compositions was evaluated. Particularly, varying exemplary film forming oral care compositions (1)-(9) were evaluated for their respective stability under accelerated aging conditions. Each of the oral care compositions (1)-(9) were prepared by combining the ingredients/components according to Table 1. It should be noted that a film forming oral care composition including 15% active oxygen (i.e., 30 wt % of a 50% hydrogen peroxide solution) and about 18 wt % of the acrylates/octylacrylamide copolymer was prepared, but was not stable due to the amount of water present. It should be further be noted that a film forming oral care composition including 10% active oxygen (i.e., 20 wt % of a 50% hydrogen peroxide solution) and about 18 wt % of the acrylates/octylacrylamide copolymer was prepared, but was not stable due to the amount of water present.

TABLE 1
Oral Care Compositions (1)-(9)

| Ingredient/ | 1 | 2 | 3 | 4 | 5 | 6 | 7 | 8 | 9 |
|---|---|---|---|---|---|---|---|---|---|
| Component | (wt %) | (wt %) | (wt %) | (wt %) | (wt %) | (wt %) | (wt %) | (wt %) | (wt %) |
| Anhydrous | 89.94 | 73.71 | 82.0 | 76.0 | 79.94 | 70.0 | 71.94 | 60.0 | 61.8 |
| Ethanol | | | | | | | | | |
| 50% Hydrogen | 6.0 | 6.0 | 6.0 | 6.0 | 12.0 | 12.0 | 20.0 | 20.0 | 30.0 |
| Peroxide | | | | | | | | | |
| Solution | | | | | | | | | |
| Acrylates/ | 1.0 | 5.0 | 10.0 | 18.0 | 5.0 | 18.0 | 5.0 | 18.0 | 5.0 |
| Octylacrylamide | | | | | | | | | |
| Copolymer | | | | | | | | | |
| Sodium | 0.20 | 1.0 | 0.155 | — | 0.20 | — | 0.20 | 0.155 | 0.20 |
| Bicarbonate | | | | | | | | | |
| Deionized Water | 2.86 | 14.29 | 1.845 | — | 2.86 | — | 2.86 | 1.845 | 3.0 |
| Total | 100 | 100 | 100 | 100 | 100 | 100 | 100 | 100 | 100 |
| Components | | | | | | | | | |

The stability of each of the oral care compositions (1)-(9) was evaluated with respect to color/appearance and active oxygen, which represents the ability of the oral care composition for preventing or inhibiting degradation of the hydrogen peroxide. To evaluate the active oxygen, each of the oral care compositions (1)-(9) was placed in a respective glass jar and exposed to an environment maintained at the particular condition (i.e., temperature, relative humidity, time) indicated in Table 2. After the respective times of each of the studies elapsed, each of the oral care compositions (1)-(9) was removed from the controlled environment and allowed to cool or warm in a monitored stability chamber maintained at about 25° C. After cooling or warming to about 25° C., the active oxygen was measured, and the respective oral care composition was observed/evaluated visually. The results of the measured active oxygen are summarized in Table 2.

TABLE 2
Active Oxygen for Oral Care Compositions (1)-(9)

| | 30° C./ | 30° C./ | 40° C./ | 40° C./ | 40° C./ |
|---|---|---|---|---|---|
| | 65% RH | 65% RH | 75% RH | 75% RH | 75% RH |
| | Initial | 3-mos. | 1-mo. | 2-mos. | 3-mos. |
| # | (%) | (%) | (%) | (%) | (%) |
| 1 | 3.10 | 3.00 | 3.10 | 3.10 | 2.80 |
| 2 | 3.20 | 3.10 | 3.20 | 3.00 | 3.00 |
| 3 | 3.10 | 3.00 | 3.10 | 3.00 | 3.00 |
| 4 | 3.10 | 3.00 | 3.00 | 2.90 | 2.80 |
| 5 | 6.20 | 6.20 | 6.10 | 6.10 | 6.10 |
| 6 | 6.10 | 6.10 | 6.00 | 6.00 | 5.70 |
| 7 | 10.20 | 10.20 | 10.20 | 10.10 | 10.10 |
| 8 | 10.60 | 10.34 | 10.34 | 10.29 | 10.02 |
| 9 | 15.40 | 15.30 | 15.10 | 15.10 | 15.20 |

It should be appreciated that the initial active oxygen in oral care compositions (1)-(4) was about 3%, the initial active oxygen in oral care compositions (5) and (6) was about 6%, the initial active oxygen in oral care compositions (7) and (8) was about 10%, and the initial active oxygen in oral care composition (9) was about 15%. As illustrated in Table 2, it was surprisingly and unexpectedly discovered that each of the exemplary oral care compositions, which included a combination of sodium bicarbonate and relatively low amounts (e.g., less than 18 wt %) of the acrylate/octylacrylamide copolymer, was able to maintain the stability of and prevent or reduce the degradation of the peroxide contained therein. It was also surprisingly and unexpectedly observed that each of the oral care compositions including the combination of sodium bicarbonate and relatively low amounts (e.g., less than 18 wt %) of the acrylate/octylacrylamide copolymer maintained a clear, colorless liquid after being exposed to the accelerated aging conditions. The stability of each of the exemplary oral care compositions including the combination of the copolymer and the sodium bicarbonate was demonstrated under accelerated aging conditions for up to 1-month, up to 2-months, and up to 3-months.

### Example 2

The in vitro whitening efficacy or performance of oral care compositions (1), (2), and (4) was evaluated. A comparative oral care composition (10), similar to oral care compositions (1), but excluding the sodium bicarbonate, was also prepared and evaluated. Specifically, comparative oral care composition (10) included 3% active oxygen, 1.0 wt % of the acrylates/octylacrylamide copolymer, and excluded sodium bicarbonate. The whitening efficacy was evaluated using artificially stained bovine tooth blocks (incisors mounted in an acrylic resin) commercially available from Dental Product Testing Therametric Technologies, Inc. Three of the stained bovine tooth blocks with L values of 60-65 were cleaned and wiped dry. The L, a, b values were measured by a MHT SpectroShade. The tooth blocks were coated with each of the respective film forming oral care compositions (1), (2), (4), and (10) with a brush. The respective film forming oral care compositions (1), (2), (4), and (10) were air dried to allow sufficient evaporation of the ethanol, thereby forming a film including the hydrogen peroxide, sodium bicarbonate, and/or the acrylates/octylacrylamide copolymer (i.e., DERMACRYL® 79) coated on the tooth blocks. The coated blocks were immersed in artificial saliva in 12 well plates and incubated overnight at about 37° C. with about 100 rpm agitation. This allowed at least some of the hydrogen peroxide to diffuse out from the film and interact with the tooth blocks. The next morning, the tooth blocks were taken out from the artificial saliva and the remaining film was removed by wiping with ethanol. L, a, b values were measured again using SpectroShade. This procedure was repeated for each treatment with the respective film forming oral care compositions (1), (2), (4), and (10).

Whitening performance or efficacy was reported as a change of W value (ΔW) after each treatment compared to baseline value according to:

$${W}^{*} = {( {( {a}^{*} )}^{2} + {( {b}^{*} )}^{2} + {( {L}^{*} - 100 )}^{2} )}^{1 / 2} and$$

$$Δ {W}^{*} = {W}_{created}^{*} - {W}_{baseline}^{*} .$$

The results of the whitening efficacy (ΔW) are summarized in Table 3.

TABLE 3
Whitening Efficacy (ΔW) of Oral Care
Compositions (1), (2), (4), and (10)

| | No. of Treatments | | | | | | | |
|---|---|---|---|---|---|---|---|---|
| # | 0 | 1 | 2 | 3 | 4 | 5 | 6 | 7 |
| (1) | 0 | −3.0 | −4.2 | −6.3 | −6.9 | −7.8 | −8.5 | −9.4 |
| (2) | 0 | −2.9 | −3.9 | −5.9 | −6.9 | −8.0 | −8.9 | −9.4 |
| (4) | 0 | −2.8 | −3.5 | −5.8 | −6.5 | −7.6 | −7.9 | −8.7 |
| (10) | 0 | −0.3 | −1.6 | −2.3 | −2.9 | −3.7 | −4.1 | −4.6 |

As illustrated in Table 3, it was surprisingly and unexpectedly discovered that oral care composition (1), which included 3% hydrogen peroxide, 1% of the copolymer, and sodium bicarbonate, exhibited whitening efficacy similar or comparable to oral care composition (4), which included 3% hydrogen peroxide and 18% of the copolymer, and excluded sodium bicarbonate. It was also surprisingly and unexpectedly discovered that oral care composition (1), which included about 1 wt % of the copolymer, exhibited whitening efficacy similar or comparable to oral care composition (2), which included about 5 wt % of the copolymer. It was further surprisingly and unexpectedly discovered that oral care composition (10), which included 3% hydrogen peroxide and 1% of the copolymer and excluded sodium bicarbonate, exhibited reduced whitening efficacy as compared to oral care composition (1), which included sodium bicarbonate.

The in vitro whitening efficacy or performance of oral care compositions (5) and (6) was also evaluated. A comparative oral care composition (11), similar to oral care compositions (5), but excluding the sodium bicarbonate, was also prepared and evaluated. Specifically, comparative oral care composition (11) included 6% active oxygen, 5.0 wt % of the acrylates/octylacrylamide copolymer, and excluded sodium bicarbonate. The results of the whitening efficacy are summarized in Table 4.

TABLE 4
Whitening Efficacy (ΔW) of Oral
Care Compositions (5), (6), and (11)

No. of Treatments

| # | 0 | 1 | 2 | 3 |
|---|---|---|---|---|
| (5) | 0 | −2.3 | −3.5 | −5.1 |
| (6) | 0 | −3.0 | −4.5 | −5.5 |
| (11) | 0 | −2.1 | −2.6 | −2.2 |

As illustrated in Table 4, it was surprisingly and unexpectedly discovered that oral care composition (5), which included 6% hydrogen peroxide, 5% of the copolymer, and sodium bicarbonate, exhibited whitening efficacy similar or comparable to oral care composition (6), which included 6% hydrogen peroxide and 18% of the copolymer, and excluded sodium bicarbonate. It was also surprisingly and unexpectedly discovered that oral care composition (11), which included 6% hydrogen peroxide and 5% of the copolymer and excluded sodium bicarbonate, exhibited reduced whitening efficacy as compared to oral care composition (5), which included sodium bicarbonate. The observed whitening efficacy demonstrates and supports a synergistic interaction between the sodium bicarbonate and the hydrogen peroxide in the oral care composition.

The foregoing demonstrates and supports a synergistic interaction between the sodium bicarbonate and the hydrogen peroxide in the oral care composition for improved whitening efficacy and/or improved preservation of the hydrogen peroxide. Without being bound by theory, it is believed that the improved whitening efficacy, when combining the hydrogen peroxide and the sodium bicarbonate in an oral care composition having relatively low concentrations (e.g., less than 18 wt %, less than 15 wt %, less than 10 wt %) of the polymer (e.g., the acrylates/octylacrylamide copolymer), is attributed to the improved preservation of hydrogen peroxide and/or the reduced degradation of the hydrogen peroxide.

The present disclosure has been described with reference to exemplary implementations. Although a limited number of implementations have been shown and described, it will be appreciated by those skilled in the art that changes may be made in these implementations without departing from the principles and spirit of the preceding detailed description. It is intended that the present disclosure be construed as including all such modifications and alterations insofar as they come within the scope of the appended claims or the equivalents thereof.

1. A film forming oral care composition for whitening teeth, the film forming oral care composition comprising:
a whitening agent, wherein the whitening agent comprises an oxidizing agent present in an amount of from greater than 0 wt. % to about 10 wt. % or less, based on the total weight of the oral care composition;
an alkali metal carbonate or bicarbonate;
a copolymer of acrylates and octylacrylamide, wherein the copolymer is present in an amount less than 18 wt. %, based on the total weight of the oral care composition; and
an orally acceptable solvent.

2. The film forming oral care composition of claim 1, wherein the oxidizing agent comprises a peroxide compound, preferably, the peroxide compound comprises hydrogen peroxide.

3. The film forming oral care composition of claim 1, wherein the copolymer of acrylates and octylacrylamide comprises 2-propenoic acid, 2-methyl-, 2-methylpropyl ester, 2-propenoic acid, N-(1,1,3,3-tetramethylbutyl)-2-propenamide.

4. The film forming oral care composition of claim 1, wherein the copolymer of acrylates and octylacrylamide is present in an amount of from about 1 wt. % to less than 18 wt. %, preferably, about 1 wt. % to about 10 wt. %, more preferably, about 1 wt. % to about 5 wt. %, based on the total weight of the oral care composition.

5. The film forming oral care composition of claim 1, wherein the alkali metal carbonate or bicarbonate comprises one or more of sodium carbonate, potassium carbonate, sodium bicarbonate, potassium bicarbonate, or a combination thereof, preferably, the alkali metal carbonate or bicarbonate comprises sodium bicarbonate.

6. The film forming oral care composition of claim 1, wherein the alkali metal carbonate or bicarbonate is present in an amount sufficient to interact synergistically with the whitening agent to prevent the degradation of the whitening agent, preferably, the alkali metal carbonate or bicarbonate is present in an amount from about 0.1 wt. % to about 1 wt. %, about 0.15 wt. % to about 0.8 wt. %, or about 0.2 wt. % to about 0.5 wt. %, based on the total weight of the oral care product or the film forming oral care composition thereof.

7. The film forming oral care composition of claim 1, wherein the alkali metal carbonate or bicarbonate and the whitening agent are present in a synergistic weight ratio of from about 1:3 to about 1:75, preferably, a synergistic weight ratio of about 1:3, about 1:15, about 1:19, about 1:30, about 1:50, about 1:65, or about 1:75.

8. The film forming oral care composition of claim 1, wherein the orally acceptable solvent comprises one or more of ethanol, methanol, isopropanol, butanol, water, acetone, methyl ethyl ketone, ethyl acetate, butyl acetate, cyclohexanone, cyclohexane, methylene chloride, chloroform, carbon tetrachloride, trichloroethylene, perchloroethylene, methyl glycol acetate, toluene, benzene, diethyl ether, benzyl alcohol, or the like, or a combination thereof, preferably, the orally acceptable solvent comprises ethanol.

9. The film forming oral care composition of claim 1, wherein the film forming oral care composition is free of cellulose and derivatives of cellulose.

10. The film forming oral care composition of claim 1, further comprising an additional component comprising one or more of an anti-caries agent, an anti-calculus agent, an anti-inflammatory agent, a desensitizing agent, a viscosity modifier, a thickening agent, a diluent, a surface active agent, an orally acceptable surfactant, a pH modifying agent, a therapeutic agent, a humectant, a mouth feel agent, an enzyme, a sweetening agent, a flavor agent, preservatives, vitamins, pigments, or a combination thereof.

11. The film forming oral care composition of claim 1, wherein the whitening agent further comprises a whitening pigment.

12. The film forming oral care composition of claim 1, wherein the film forming oral care composition comprises a viscosity measured at about 25° C. of from about 1 cP to less than 60 cP, preferably, about 1 cP to about 15 cP, more preferably about 2 cP to about 11 cP.

13. The film forming oral care composition of claim 1, wherein the film forming oral care composition consists of the whitening agent, the alkali metal carbonate or bicarbonate, the copolymer of acrylates and octylacrylamide, the orally acceptable solvent, and optionally, the additional component.

14. A film prepared from the film forming oral care composition of claim 1.

15. A method for whitening teeth, the method comprising:
applying the film forming oral care composition of claim 1 to surfaces of the teeth; and
evaporating at least a portion of the orally acceptable solvent to form a film on the surfaces of the teeth.
